(19) United States
(12) Patent Application Publication
(10) Pub. No.: US 2023/0407278 A1
(43) Pub. Date: Dec. 21, 2023

(54) COMPOSITIONS AND METHODS FOR CAS9 MOLECULES WITH IMPROVED GENE EDITING PROPERTIES

(71) Applicant: BOARD OF REGENTS, THE UNIVERSITY OF TEXAS SYSTEM, Austin, TX (US)

(72) Inventors: David Taylor, Austin, TX (US); Kenneth A. Johnson, Austin, TX (US); Jack P. K. Bravo, Austin, TX (US); Tyler L. Dangerfield, Austin, TX (US)

(21) Appl. No.: 18/186,443

(22) Filed: Mar. 20, 2023

Related U.S. Application Data
(60) Provisional application No. 63/321,419, filed on Mar. 18, 2022.
(60) Provisional application No. 63/330,593, filed on Apr. 13, 2022.

(51) Int. Cl. C12N 9/22; C12N 15/10

(52) U.S. Cl. CPC C12N 9/22; C12N 15/102; C12N 2310/20

(57) ABSTRACT

Disclosed herein are methods and compositions relating to a mutated version of Cas9. This mutation or mutations can be in the Rec3 clamp of Cas9. The mutated Cas9 can have advantages compared Cas9s which don't have mutations in the Rec3 clamp.

### CROSS-REFERENCE TO RELATED APPLICATIONS

This application claims benefit of U.S. Provisional Application No. 63/321,419, filed Mar. 18, 2022, and U.S. Provisional Application 63/330,593, filed Apr. 13, 2022, both of which are incorporated herein by reference in their entirety.

### GOVERNMENT SUPPORT CLAUSE

This invention was made with government support under Grant no. R01 AI163336 awarded by the National Institutes of Health. The government has certain rights in the invention.”

### SEQUENCE LISTING STATEMENT

The specification further incorporates by reference the Sequence Listing submitted on Mar. 20, 2023 via the U.S.P.T.O.'s electronic filing system (EFS). The Sequence Listing .xml file, identified as “10046-479US1 2023_03_20 Sequence Listing.xml” is 5,433 bytes and was created on Mar. 20, 2023. The Sequence Listing, electronically filed herewith, does not extend beyond the scope of the specification, and does not contain new matter.

### BACKGROUND

Genome engineering refers to the strategies and techniques for the targeted, specific modification of the genetic information (genome) of living organisms. Genome engineering is a very active field of research because of the wide range of possible applications, particularly in the areas of human health. For example, genome engineering can be used to alter (e.g., correct or knock-out) a gene carrying a harmful mutation or to explore the function of a gene. Early technologies developed to insert a transgene into a living cell were often limited by the random nature of the insertion of the new sequence into the genome. Random insertions into the genome may result in disrupting normal regulation of neighboring genes leading to severe unwanted effects. Furthermore, random integration technologies offer little reproducibility, as there is no guarantee that the sequence would be inserted at the same place in two different cells.

Clustered regularly interspaced short palindromic repeats (CRISPR) and CRISPR-associated system (CRISPR/-Cas9) is a popular tool for genome editing. However, use of CRISPR-Cas9 as a programmable genome editing tool is hindered by off-target DNA cleavage (Cong et al., 2013; Doudna, 2020; Fu et al., 2013; Jinek et al., 2013), and the underlying mechanisms by which Cas9 recognizes mismatches are poorly understood (Kim et al., 2019; Liu et al., 2020; Slaymaker and Gaudelli, 2021). Although Cas9 variants with greater discrimination against mismatches have been designed (Chen et al., 2017; Kleinstiver et al., 2016; Slaymaker et al., 2016), these suffer from significantly reduced on-target DNA cleavage rates (Kim et al., 2020; Liu et al., 2020).

While certain functions of Cas9 are linked to (but not necessarily fully determined by) their specific domains, there has been a lack of understanding of which domains correlate with changes in efficiency, processivity, and specificity of Cas9. For therapeutic applications of CRISPR-Cas9 to reach their full potential, it is necessary to minimize off-target DNA cleavage (Cong et al., 2013; Fu et al., 2013; Jinek et al., 2013) and to maximize processivity and efficiency.

What is needed in the art are variant Cas9 molecules with improved gene editing properties as compared to native Cas9 molecules.

### SUMMARY

Disclosed herein is a method of increasing fidelity, specificity, and/or speed of processivity in a functional Cas9 molecule, the method comprising mutating one or more amino acid residues within a clamp region of Cas9 Recognition Lobe (REC3 clamp), wherein the REC3 clamp is 80% or more identical to SEQ ID NO: 1, and further wherein the one or more mutations increase fidelity, specificity, and/or speed of processivity of the Cas9 molecule.

Also disclosed is a method of performing gene editing, the method comprising contacting a target site with a functional Cas9 molecule, wherein the Cas9 molecule comprises a REC3 clamp, wherein the REC3 clamp is 80% or more identical to SEQ ID NO: 1, and further wherein the REC3 clamp comprises one or more amino acid mutations which increase fidelity, specificity, and/or speed of processivity compared to a REC3 clamp without said one or more mutations.

Further disclosed is a method of treating a subject with a disease or disorder which is treatable with gene editing, the method comprising contacting a target site of one or more genes in need of editing within the genome of the subject with a functional Cas9 molecule comprising a REC3 clamp, wherein the REC3 clamp is 80% or more identical to SEQ ID NO: 1, and further wherein the REC3 clamp comprises one or more amino acid mutations which increase fidelity, specificity, and/or speed of processivity compared to a REC3 clamp without said one or more mutations; wherein said Cas9 molecule edits one or more genes in a manner which effectively treats said disease or disorder.

Disclosed is a method of modifying an organism to produce a non-naturally occurring product, or a naturally occurring product in a non-naturally occurring amount, the method comprising contacting a target site of one or more genes within the genome of the organism with a functional Cas9 molecule comprising a REC3 clamp, wherein the REC3 clamp is 80% or more identical to SEQ ID NO: 1, and further wherein the REC3 clamp comprises one or more amino acid mutations which increase fidelity, specificity, and/or speed of processivity compared to a REC3 clamp without said one or more mutations; wherein said Cas9 molecule edits one or more genes of interest so that the organism produces a non-naturally occurring product, or a naturally occurring product in a non-naturally occurring amount.

### DETAILED DESCRIPTION

### General Definitions

Unless defined otherwise, all technical and scientific terms used herein have the same meaning as commonly understood to one of ordinary skill in the art to which this disclosure belongs.

Ranges can be expressed herein as from “about” one particular value, and/or to “about” another particular value. By “about” is meant within 10% of the value, e.g., within 9, 8, 7, 6, 5, 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. Throughout the description and claims of this specification the word “comprise” and other forms of the word, such as “comprising” and “comprises,” means including but not limited to, and is not intended to exclude, for example, other additives, components, integers, or steps.

As used in the specification and claims, the singular form “a”, “an”, and “the” include plural references unless the context clearly dictates otherwise. For example, the term “an agent” includes a plurality of agents, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

As used herein, “nucleic acid” means a polynucleotide and includes a single or a double-stranded polymer of deoxyribonucleotide or ribonucleotide bases. Nucleic acids may also include fragments and modified nucleotides. Thus, the terms “polynucleotide”, “nucleic acid sequence”, “nucleotide sequence” and “nucleic acid fragment” are used interchangeably to denote a polymer of RNA and/or DNA and/or RNA-DNA that is single- or double-stranded, optionally comprising synthetic, non-natural, or altered nucleotide bases. On occasion double-stranded DNA will be referred to “duplex DNA” or “dsDNA”. Nucleotides (usually found in their 5′ -monophosphate form) are referred to by their single letter designation as follows: “A” for adenosine or deoxyadenosine (for RNA or DNA, respectively), “C” for cytosine or deoxycytosine, “G” for guanosine or deoxyguanosine, “U” for uridine, “T” for deoxythymidine, “R” for purines (A or G), “Y” for pyrimidines (C or T), “K” for G or T, “H” for A or C or T, “I” for inosine, and “N” for any nucleotide.

The term “genome” as it applies to a prokaryotic and eukaryotic cell or organism cells encompasses not only chromosomal DNA found within the nucleus, but organelle DNA found within subcellular components (e.g., mitochondria, or plastid) of the cell.

“Open reading frame” is abbreviated ORF.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 80% sequence identity, or 90% sequence identity, up to and including 100% sequence identity (i.e., fully complementary) with each other.

The term “stringent conditions” or “stringent hybridization conditions” includes reference to conditions under which a probe will selectively hybridize to its target sequence in an in vitro hybridization assay. Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length. Typically, stringent conditions will be those in which the salt concentration is less than about 1.5 M Na ion, typically about 0.01 to 1.0 M Na ion concentration (or other salt(s)) at pH 7.0 to 8.3, and at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide Exemplary low stringency conditions include hybridization with a buffer solution of 30 to 35% formamide, 1 M NaCl, 1% SDS (sodium dodecyl sulphate) at 37° C., and a wash in 1X to 2X SSC (20X SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5X to 1X SSC at 55 to 60° C. Exemplary high stringency conditions include hybridization in 50% formamide, 1 M NaCl, 1% SDS at 37° C., and a wash in 0.1X SSC at 60 to 65° C.

By “homology” is meant DNA sequences that are similar. For example, a “region of homology to a genomic region” that is found on the donor DNA is a region of DNA that has a similar sequence to a given “genomic region” in the cell or organism genome. A region of homology can be of any length that is sufficient to promote homologous recombination at the cleaved target site. For example, the region of homology can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800, 5-2900, 5-3000, 5-3100 or more bases in length such that the region of homology has sufficient homology to undergo homologous recombination with the corresponding genomic region.

“Sufficient homology” indicates that two polynucleotide sequences have sufficient structural similarity to act as substrates for a homologous recombination reaction. The structural similarity includes overall length of each polynucleotide fragment, as well as the sequence similarity of the polynucleotides. Sequence similarity can be described by the percent sequence identity over the whole length of the sequences, and/or by conserved regions comprising localized similarities such as contiguous nucleotides having 100% sequence identity, and percent sequence identity over a portion of the length of the sequences.

As used herein, a “genomic region” is a segment of a chromosome in the genome of a cell that is present on either side of the target site or, alternatively, also comprises a portion of the target site. The genomic region can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800. 5-2900, 5-3000, 5-3100 or more bases such that the genomic region has sufficient homology to undergo homologous recombination with the corresponding region of homology.

As used herein, “homologous recombination” (HR) includes the exchange of DNA fragments between two DNA molecules at the sites of homology. The frequency of homologous recombination is influenced by a number of factors. Different organisms vary with respect to the amount of homologous recombination and the relative proportion of homologous to non-homologous recombination. Generally, the length of the region of homology affects the frequency of homologous recombination events; the longer the region of homology, the greater the frequency. The length of the homology region needed to observe homologous recombination is also species-variable. In many cases, at least 5 kb of homology has been utilized, but homologous recombination has been observed with as little as 25-50 bp of homology. See, for example, Singer et al. , (1982) Cell 31 :25-33; Shen and Huang, (1986) Genetics 112:441-57; Watt et al. , (1985) Proc. Natl. Acad. Sci. USA 82:4768-72, Sugawara and Haber, (1992)Mol Cell Biol 12:563-75, Rubnitz and Subramani, (1984) o/ Cell Biol 4:2253-8; Ayares et al. , (1986) Proc. Natl. Acad. Sci. USA 83:5199-203; Liskay et al. , (1987) Genetics 115: 161-7.

“Sequence identity” or “identity” in the context of nucleic acid or polypeptide sequences refers to the nucleic acid bases or amino acid residues in two sequences that are the same when aligned for maximum correspondence over a specified comparison window.

The term “percentage of sequence identity” refers to the value determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. Useful examples of percent sequence identities include, but are not limited to, 50%, 55%, 60%, 65%, 70%, 75%, 80%, 85%, 90%, or 95%, or any percentage from 50% to 100%. These identities can be determined using any of the programs described herein.

Sequence alignments and percent identity or similarity calculations may be determined using a variety of comparison methods designed to detect homologous sequences including, but not limited to, the MegAlign™ program of the LASERGENE bioinformatics computing suite (DNASTAR Inc., Madison, WI). Within the context of this application it will be understood that where sequence analysis software is used for analysis, that the results of the analysis will be based on the “default values” of the program referenced, unless otherwise specified. As used herein “default values” will mean any set of values or parameters that originally load with the software when first initialized.

The “Clustal V method of alignment” corresponds to the alignment method labeled Clustal V (described by Higgins and Sharp, (1989) CABIOS 5:151-153; Higgins et al., (1992) Comput Appl Biosci 8: 189-191) and found in the MegAlign™ program of the LASERGENE bioinformatics computing suite (DNASTAR Inc., Madison, WI). For multiple alignments, the default values correspond to GAP PENALTY=10 and GAP LENGTH PENALTY=10. Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences using the Clustal V program, it is possible to obtain a “percent identity” by viewing the “sequence distances” Table in the same program. The “Clustal W method of alignment” corresponds to the alignment method labeled Clustal W (described by Higgins and Sharp, (1989) CABIOS 5:151-153; Higgins et al ., (1992) Comput Appl Biosci 8:189-191) and found in the MegAlign™ v6.1 program of the LASERGENE bioinformatics computing suite (DNASTAR Inc., Madison, WI). Default parameters for multiple alignment (GAP PENALTY=10, GAP LENGTH PENALTY=0.2, Delay Divergen Seqs (%)=30, DNA Transition Weight=0.5, Protein Weight Matrix=Gonnet Series, DNA Weight Matrix=IUB). After alignment of the sequences using the Clustal W program, it is possible to obtain a “percent identity” by viewing the “sequence distances” Table in the same program. Unless otherwise stated, sequence identity/similarity values provided herein refer to the value obtained using GAP Version 10 (GCG, Accelrys, San Diego, CA) using the following parameters:% identity and % similarity for a nucleotide sequence using a gap creation penalty weight of 50 and a gap length extension penalty weight of 3, and the nwsgapdna.cmp scoring matrix; % identity and % similarity for an amino acid sequence using a GAP creation penalty weight of 8 and a gap length extension penalty of 2, and the BLOSUM62 scoring matrix (Henikoff and Henikoff, (1989) Proc. Natl. Acad. Sci. USA 89: 10915). GAP uses the algorithm of Needleman and Wunsch, (1970) J Mol Biol 48:443-53, to find an alignment of two complete sequences that maximizes the number of matches and minimizes the number of gaps. GAP considers all possible alignments and gap positions and creates the alignment with the largest number of matched bases and the fewest gaps, using a gap creation penalty and a gap extension penalty in units of matched bases.

“BLAST” is a searching algorithm provided by the National Center for Biotechnology Information (NCBI) used to find regions of similarity between biological sequences. The program compares nucleotide or protein sequences to sequence databases and calculates the statistical significance of matches to identify sequences having sufficient similarity to a query sequence such that the similarity would not be predicted to have occurred randomly. BLAST reports the identified sequences and their local alignment to the query sequence. It is well understood by one skilled in the art that many levels of sequence identity are useful in identifying polypeptides from other species or modified naturally or synthetically wherein such polypeptides have the same or similar function or activity. Useful examples of percent identities include, but are not limited to, 50%, 55%, 60%, 65%, 70%, 75%, 80%, 85%, 90% or 95%, or any percentage from 50% to 100%. Indeed, any amino acid identity from 50% to 100% may be useful in describing the present disclosure, such as 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%.

Polynucleotide and polypeptide sequences, variants thereof, and the structural relationships of these sequences can be described by the terms “homology”, “homologous”, “substantially identical”, “substantially similar” and “corresponding substantially” which are used interchangeably herein. These refer to polypeptide or nucleic acid sequences wherein changes in one or more amino acids or nucleotide bases do not affect the function of the molecule, such as the ability to mediate gene expression or to produce a certain phenotype. These terms also refer to modification(s) of nucleic acid sequences that do not substantially alter the functional properties of the resulting nucleic acid relative to the initial, unmodified nucleic acid. These modifications include deletion, substitution, and/or insertion of one or more nucleotides in the nucleic acid fragment. Substantially similar nucleic acid sequences encompassed may be defined by their ability to hybridize (under moderately stringent conditions, e.g., 0.5X SSC, 0.1% SDS, 60° C.) with the sequences exemplified herein, or to any portion of the nucleotide sequences disclosed herein and which are functionally equivalent to any of the nucleic acid sequences disclosed herein. Stringency conditions can be adjusted to screen for moderately similar fragments, such as homologous sequences from distantly related organisms to highly similar fragments, such as genes that duplicate functional enzymes from closely related organisms. Post-hybridization washes determine stringency conditions.

A “centimorgan” (cM) or “map unit” is the distance between two polynucleotide sequences, linked genes, markers, target sites, loci, or any pair thereof, wherein 1% of the products of meiosis are recombinant. Thus, a centimorgan is equivalent to a distance equal to a 1% average recombination frequency between the two linked genes, markers, target sites, loci, or any pair thereof.

An “isolated” or “purified” nucleic acid molecule, polynucleotide, polypeptide, or protein, or biologically active portion thereof, is substantially or essentially free from components that normally accompany or interact with the polynucleotide or protein as found in its naturally occurring environment. Thus, an isolated or purified polynucleotide or polypeptide or protein is substantially free of other cellular material, or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. Isolated polynucleotides may be purified from a cell in which they naturally occur. Conventional nucleic acid purification methods known to skilled artisans may be used to obtain isolated polynucleotides. The term also embraces recombinant polynucleotides and chemically synthesized polynucleotides.

The term “fragment” refers to a contiguous set of nucleotides or amino acids. In one embodiment, a fragment is 2, 3, 4, 5, 6, 7 8, 9, 10, 11, 12, 13, 14, 15, 16, 17, 18, 19, 20, or greater than 20 contiguous nucleotides. In one embodiment, a fragment is 2, 3, 4, 5, 6, 7 8, 9, 10, 11, 12, 13, 14, 15, 16, 17, 18, 19, 20, or greater than 20 contiguous amino acids. A fragment may or may not exhibit the function of a sequence sharing some percent identity over the length of said fragment.

The terms “fragment that is functionally equivalent” and “functionally equivalent fragment” are used interchangeably herein. These terms refer to a portion or subsequence of an isolated nucleic acid fragment or polypeptide that displays the same activity or function as the longer sequence from which it derives. In one example, the fragment retains the ability to alter gene expression or produce a certain phenotype whether or not the fragment encodes an active protein. For example, the fragment can be used in the design of genes to produce the desired phenotype in a modified organism. Genes can be designed for use in suppression by linking a nucleic acid fragment, whether or not it encodes an active enzyme, in the sense or antisense orientation relative to a native promoter sequence.

“Gene” includes a nucleic acid fragment that expresses a functional molecule such as, but not limited to, a specific protein, including regulatory sequences preceding (5′ noncoding sequences) and following (3′ non-coding sequences) the coding sequence. “Native gene” refers to a gene as found in its natural endogenous location with its own regulatory sequences.

By the term “endogenous” it is meant a sequence or other molecule that naturally occurs in a cell or organism. In one aspect, an endogenous polynucleotide is normally found in the genome of a cell; that is, not heterologous.

An “allele” is one of several alternative forms of a gene occupying a given locus on a chromosome. When all the alleles present at a given locus on a chromosome are the same, that organism is homozygous at that locus. If the alleles present at a given locus on a chromosome differ, that organism is heterozygous at that locus.

“Coding sequence” refers to a polynucleotide sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include, but are not limited to, promoters, translation leader sequences, 5′ untranslated sequences, 3′ untranslated sequences, introns, polyadenylation target sequences, RNA processing sites, effector binding sites, and stem-loop structures.

A “mutated gene” is a gene that has been altered through human intervention. Such a “mutated gene” has a sequence that differs from the sequence of the corresponding non-mutated gene by at least one nucleotide addition, deletion, or substitution. In certain embodiments of the disclosure, the mutated gene comprises an alteration that results from a guide polynucleotide/Cas endonuclease system as disclosed herein. A mutated organism is an organism comprising a mutated gene.

As used herein, a “targeted mutation” is a mutation in a gene (referred to as the target gene), including a native gene, that was made by altering a target sequence within the target gene using any method known to one skilled in the art, including a method involving a guided Cas endonuclease system as disclosed herein.

The terms “knock-out”, “gene knock-out” and “genetic knock-out” are used interchangeably herein. A knock-out represents a DNA sequence of a cell that has been rendered partially or completely inoperative by targeting with a Cas protein; for example, a DNA sequence prior to knock-out could have encoded an amino acid sequence, or could have had a regulatory function (e.g., promoter).

The terms “knock-in”, “gene knock-in, “gene insertion” and “genetic knock-in” are used interchangeably herein. A knock-in represents the replacement or insertion of a DNA sequence at a specific DNA sequence in cell by targeting with a Cas protein (for example by homologous recombination (HR), wherein a suitable donor DNA polynucleotide is also used) examples of knock-ins are a specific insertion of a heterologous amino acid coding sequence in a coding region of a gene, or a specific insertion of a transcriptional regulatory element in a genetic locus.

By “domain” it is meant a contiguous stretch of nucleotides (that can be RNA, DNA, and/or RNA-DNA-combination sequence) or amino acids.

The term “conserved domain” or “motif” means a set of polynucleotides or amino acids conserved at specific positions along an aligned sequence of evolutionarily related proteins. While amino acids at other positions can vary between homologous proteins, amino acids that are highly conserved at specific positions indicate amino acids that are essential to the structure, the stability, or the activity of a protein. Because they are identified by their high degree of conservation in aligned sequences of a family of protein homologues, they can be used as identifiers, or “signatures”, to determine if a protein with a newly determined sequence belongs to a previously identified protein family.

A “codon-modified gene” or “codon-preferred gene” or “codon-optimized gene” is a gene having its frequency of codon usage designed to mimic the frequency of preferred codon usage of the host cell.

An “optimized” polynucleotide is a sequence that has been optimized for improved expression in a particular heterologous host cell.

A “promoter” is a region of DNA involved in recognition and binding of RNA polymerase and other proteins to initiate transcription. The promoter sequence consists of proximal and more distal upstream elements, the latter elements often referred to as enhancers.

An “enhancer” is a DNA sequence that can stimulate promoter activity and may be an innate element of the promoter or a heterologous element inserted to enhance the level or tissue- specificity of a promoter. Promoters may be derived in their entirety from a native gene or be composed of different elements derived from different promoters found in nature, and/or comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity.

Promoters that cause a gene to be expressed in most cell types at most times are commonly referred to as “constitutive promoters”. The term “inducible promoter” refers to a promoter that selectively express a coding sequence or functional RNA in response to the presence of an endogenous or exogenous stimulus, for example by chemical compounds (chemical inducers) or in response to environmental, hormonal, chemical, and/or developmental signals. Inducible or regulated promoters include, for example, promoters induced or regulated by light, heat, stress, flooding or drought, salt stress, osmotic stress, phytohormones, wounding, or chemicals such as ethanol, abscisic acid (ABA), jasmonate, salicylic acid, or safeners.

“Translation leader sequence” refers to a polynucleotide sequence located between the promoter sequence of a gene and the coding sequence. The translation leader sequence is present in the mRNA upstream of the translation start sequence. The translation leader sequence may affect processing of the primary transcript to mRNA, mRNA stability or translation efficiency. Examples of translation leader sequences have been described (e.g., Turner and Foster, (1995) Mol Biotechnol 3:225-236).

“3′ non-coding sequences”, “transcription terminator” or “termination sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

“RNA transcript” refers to the product resulting from RNA polymerase-catalyzed transcription of a DNA sequence. When the RNA transcript is a perfect complimentary copy of the DNA sequence, it is referred to as the primary transcript or pre-mRNA. An RNA transcript is referred to as the mature RNA or mRNA when it is a RNA sequence derived from post-transcriptional processing of the primary transcript pre-mRNA. “Messenger RNA” or “mRNA” refers to the RNA that is without introns and that can be translated into protein by the cell.

“cDNA” refers to a DNA that is complementary to, and synthesized from, an mRNA template using the enzyme reverse transcriptase. The cDNA can be single-stranded or converted into double-stranded form using the Klenow fragment of DNA polymerase I. “Sense” RNA refers to RNA transcript that includes the mRNA and can be translated into protein within a cell or in vitro. “Antisense RNA” refers to an RNA transcript that is complementary to all or part of a target primary transcript or mRNA, and that blocks the expression of a target gene (see, e.g., U.S. Pat. No. 5,107,065). The complementarity of an antisense RNA may be with any part of the specific gene transcript, i.e., at the 5′ non-coding sequence, 3′ non-coding sequence, introns, or the coding sequence. “Functional RNA” refers to antisense RNA, ribozyme RNA, or other RNA that may not be translated but yet has an effect on cellular processes. The terms “complement” and “reverse complement” are used interchangeably herein with respect to mRNA transcripts, and are meant to define the antisense RNA of the message.

The term “genome” refers to the entire complement of genetic material (genes and non-coding sequences) that is present in each cell of an organism, or virus or organelle; and/or a complete set of chromosomes inherited as a (haploid) unit from one parent.

The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., the coding sequence is under the transcriptional control of the promoter). Coding sequences can be operably linked to regulatory sequences in a sense or antisense orientation. In another example, the complementary RNA regions can be operably linked, either directly or indirectly, 5′ to the target mRNA, or 3′ to the target mRNA, or within the target mRNA, or a first complementary region is 5′ and its complement is 3′ to the target mRNA.

Generally, “host” refers to an organism or cell into which a heterologous component (polynucleotide, polypeptide, other molecule, cell) has been introduced. As used herein, a “host cell” refers to an in vivo or in vitro eukaryotic cell, prokaryotic cell (e.g., bacterial or archaeal cell), or cell from a multicellular organism (e.g., a cell line) cultured as a unicellular entity, into which a heterologous polynucleotide or polypeptide has been introduced. In some embodiments, the cell is selected from the group consisting of: an archaeal cell, a bacterial cell, a eukaryotic cell, a eukaryotic single-cell organism, a somatic cell, a germ cell, a stem cell, a plant cell, an algal cell, an animal cell, in invertebrate cell, a vertebrate cell, a fish cell, a frog cell, a bird cell, an insect cell, a mammalian cell, a pig cell, a cow cell, a goat cell, a sheep cell, a rodent cell, a rat cell, a mouse cell, a non-human primate cell, and a human cell. In some cases, the cell is in vitro. In some cases, the cell is in vivo.

The term “recombinant” refers to an artificial combination of two otherwise separated segments of sequence, e.g., by chemical synthesis, or manipulation of isolated segments of nucleic acids by genetic engineering techniques.

The terms “plasmid”, “vector” and “cassette” refer to a linear or circular extra chromosomal element often carrying genes that are not part of the central metabolism of the cell, and usually in the form of double-stranded DNA. Such elements may be autonomously replicating sequences, genome integrating sequences, phage, or nucleotide sequences, in linear or circular form, of a single- or double-stranded DNA or RNA, derived from any source, in which a number of nucleotide sequences have been joined or recombined into a unique construction which is capable of introducing a polynucleotide of interest into a cell.

“Transformation cassette” refers to a specific vector comprising a gene and having elements in addition to the gene that facilitates transformation of a particular host cell. “Expression cassette” refers to a specific vector comprising a gene and having elements in addition to the gene that allow for expression of that gene in a host.

The terms “recombinant DNA molecule”, “recombinant DNA construct”, “expression construct”, “construct”, and “recombinant construct” are used interchangeably herein. A recombinant DNA construct comprises an artificial combination of nucleic acid sequences, e.g., regulatory and coding sequences that are not all found together in nature. For example, a recombinant DNA construct may comprise regulatory sequences and coding sequences that are derived from different sources, or regulatory sequences and coding sequences derived from the same source but arranged in a manner different than that found in nature. Such a construct may be used by itself or may be used in conjunction with a vector. If a vector is used, then the choice of vector is dependent upon the method that will be used to introduce the vector into the host cells as is well known to those skilled in the art. For example, a plasmid vector can be used. The skilled artisan is well aware of the genetic elements that must be present on the vector in order to successfully transform, select and propagate host cells. The skilled artisan will also recognize that different independent transformation events may result in different levels and patterns of expression (Jones et al., (1985) EMBO J 4:2411-2418; De Almeida et al., (1989) Mol Gen Genetics 218:78-86), and thus that multiple events are typically screened in order to obtain lines displaying the desired expression level and pattern. Such screening may be accomplished standard molecular biological, biochemical, and other assays including Southern analysis of DNA, Northern analysis of mRNA expression, PCR, real time quantitative PCR (qPCR), reverse transcription PCR (RT-PCR), immunoblotting analysis of protein expression, enzyme or activity assays, and/or phenotypic analysis.

The term “heterologous” refers to the difference between the original environment, location, or composition of a particular polynucleotide or polypeptide sequence and its current environment, location, or composition. As used herein, “heterologous” in reference to a sequence can refer to a sequence that originates from a different species, variety, foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. Alternatively, one or more regulatory region(s) and/or a polynucleotide provided herein may be entirely synthetic. In another example, a target polynucleotide for cleavage by a Cas endonuclease may be of a different organism than that of the Cas endonuclease. In another example, a Cas endonuclease and guide RNA may be introduced to a target polynucleotide with an additional polynucleotide that acts as a template or donor for insertion into the target polynucleotide, wherein the additional polynucleotide is heterologous to the target polynucleotide and/or the Cas endonuclease.

The term “expression”, as used herein, refers to the production of a functional end-product (e.g., an mRNA, guide RNA, or a protein) in either precursor or mature form.

A “mature” protein refers to a post-translationally processed polypeptide (i.e., one from which any pre- or propeptides present in the primary translation product have been removed). “Precursor” protein refers to the primary product of translation of mRNA (i.e., with pre- and propeptides still present). Pre- and propeptides may be but are not limited to intracellular localization signals.

“CRISPY” (Clustered Regularly Interspaced Short Palindromic Repeats) loci refers to certain genetic loci encoding components of DNA cleavage systems, for example, used by bacterial and archaeal cells to destroy foreign DNA (Horvath and Barrangou, 2010, Science 327: 167-170; WO2007025097, published 1 Mar. 2007). A CRISPR locus can consist of a CRISPR array, comprising short direct repeats (CRISPR repeats) separated by short variable DNA sequences (called spacers), which can be flanked by diverse Cas (CRISPR-associated) genes.

As used herein, an “effector” or “effector protein” is a protein that encompasses an activity including recognizing, binding to, and/or cleaving or nicking a polynucleotide target. An effector, or effector protein, may also be an endonuclease. The “effector complex” of a CRISPR system includes Cas proteins involved in crRNA and target recognition and binding. Some of the component Cas proteins may additionally comprise domains involved in target polynucleotide cleavage.

The term “Cas protein” refers to a polypeptide encoded by a Cas (CRISPR-associated) gene. A Cas protein includes proteins encoded by a gene in a cas locus and includes adaptation molecules as well as interference molecules. An interference molecule of a bacterial adaptive immunity complex includes endonucleases. A Cas endonuclease described herein comprises one or more nuclease domains. Contemplated herein are any Cas molecules that comprise a Rec3 clamp, as described below.

As used herein, the term “Cas9 protein” refers to, but is not limited to, Cas9 proteins, Cas9-type proteins encoded by Cas9 orthologs, and synthetic proteins of Cas9. The term “Cas9 protein” as used herein refers to a wild type Cas9 protein from CRISPR-Cas9 type II B systems, Cas9 protein modifications, Cas9 protein variants, Cas9 orthologs and combinations of the same. The term “dCas9” as used herein refers to Cas9 protein variants that are Cas9 proteins deactivated by nuclease, also referred to as “catalytically inactive Cas9 protein”, or “enzymatically inactive Cas9”. Various Cas9s and their relationship with each other can be found in Gasiunas, et al. (Gasiunas G., Young, J. K., Karvelis, T. et al. A catalogue of biochemically diverse CRISPR-Cas9 orthologs. Nat Commun 11, 5512 2020, hereby incorporated by reference in its entirety for its discussion concerning Cas9 molecules). Also specifically contemplated herein are Cas9 variants such as FnCas9 (SEQ ID NO: 1). The Rec3 Clamp of FnCas9 is depicted in SEQ ID NO: 2.

A Cas protein is further defined as a functional fragment or functional variant of a native Cas protein, or a protein that shares at least 30%, between 30% and 35%, at least 35%, between 35% and 40%, at least 40%, between 40% and 45%, at least 45%, between 45% and 50%, at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, at least 500, or greater than 500 contiguous amino acids of a native Cas protein, and retains at least partial activity of the native sequence.

A “functional fragment”, “fragment that is functionally equivalent” and “functionally equivalent fragment” of a Cas endonuclease are used interchangeably herein, and refer to a portion or subsequence of the Cas endonuclease of the present disclosure in which the ability to recognize, bind to, and optionally unwind, nick or cleave (introduce a single or double strand break in) the target site is retained. The portion or subsequence of the Cas endonuclease can comprise a complete or partial (functional) peptide of any one of its domains.

The terms “functional variant”, “variant that is functionally equivalent” and “functionally equivalent variant” of a Cas endonuclease or Cas effector protein are used interchangeably herein, and refer to a variant of the Cas effector protein disclosed herein in which the ability to recognize, bind to, and optionally unwind, nick or cleave all or part of a target sequence is retained.

A Cas endonuclease may also include a multifunctional Cas endonuclease. The term “multifunctional Cas endonuclease” and “multifunctional Cas endonuclease polypeptide” are used interchangeably herein and includes reference to a single polypeptide that has Cas endonuclease functionality (comprising at least one protein domain that can act as a Cas endonuclease) and at least one other functionality, such as but not limited to, the functionality to form a complex (comprises at least a second protein domain that can form a complex with other proteins). In one aspect, the multifunctional Cas endonuclease comprises at least one additional protein domain relative (either internally, upstream (5′), downstream (3′), or both internally 5′ and 3′, or any combination thereof) to those domains typical of a Cas endonuclease.

The terms “Cascade” and “Cascade complex” are used interchangeably herein and include reference to a multi-subunit protein complex that can assemble with a polynucleotide forming a polynucleotide-protein complex (PNP). Cascade is a PNP that relies on the polynucleotide for complex assembly and stability, and for the identification of target nucleic acid sequences. Cascade functions as a surveillance complex that finds and optionally binds target nucleic acids that are complementary to a variable targeting domain of the guide polynucleotide.

The terms “cleavage-ready Cascade”, “crCascade”, “cleavage-ready Cascade complex”, “crCascade complex”, “cleavage-ready Cascade system”, “CRC” and “crCascade system”, are used interchangeably herein and include reference to a multi-subunit protein complex that can assemble with a polynucleotide forming a polynucleotide-protein complex (PNP), wherein one of the cascade proteins is a Cas endonuclease capable of recognizing, binding to, and optionally unwinding, nicking, or cleaving all or part of a target sequence.

The terms “5′ -cap” and “7-methylguanylate (m7G) cap” are used interchangeably herein. A 7-methylguanylate residue is located on the 5′ terminus of messenger RNA (mRNA) in eukaryotes. RNA polymerase II (Pol II) transcribes mRNA in eukaryotes. Messenger RNA capping occurs generally as follows: the most terminal 5′ phosphate group of the mRNA transcript is removed by RNA terminal phosphatase, leaving two terminal phosphates. A guanosine monophosphate (GMP) is added to the terminal phosphate of the transcript by a guanylyl transferase, leaving a 5′-5′ triphosphate-linked guanine at the transcript terminus. Finally, the 7-nitrogen of this terminal guanine is methylated by a methyl transferase.

The terminology “not having a 5′ -cap” herein is used to refer to RNA having, for example, a 5′-hydroxyl group instead of a 5′-cap. Such RNA can be referred to as “uncapped RNA”, for example. Uncapped RNA can better accumulate in the nucleus following transcription, since 5′ -capped RNA is subject to nuclear export. One or more RNA components herein are uncapped.

As used herein, the term “guide polynucleotide”, relates to a polynucleotide sequence that can form a complex with a Cas endonuclease, including the Cas endonuclease described herein, and enables the Cas endonuclease to recognize, optionally bind to, and optionally cleave a DNA target site. The guide polynucleotide sequence can be a RNA sequence, a DNA sequence, or a combination thereof (a RNA-DNA combination sequence).

The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA). The single guide RNA can comprise a crRNA or crRNA fragment and a tracrRNA or tracrRNA fragment of the type II CRISPR/Cas system that can form a complex with a type II Cas endonuclease, wherein said guide RNA/Cas endonuclease complex can direct the Cas endonuclease to a DNA target site, enabling the Cas endonuclease to recognize, optionally bind to, and optionally nick or cleave (introduce a single or double-strand break) the DNA target site.

The term “variable targeting domain” or “VT domain” is used interchangeably herein and includes a nucleotide sequence that can hybridize (is complementary) to one strand (nucleotide sequence) of a double strand DNA target site. The percent complementation between the first nucleotide sequence domain (VT domain) and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The variable targeting domain can be at least 12, 13, 14, 15, 16, 17, 18, 19, 20, 21, 22, 23, 24, 25, 26, 27, 28, 29 or 30 nucleotides in length. In some embodiments, the variable targeting domain comprises a contiguous stretch of 12 to 30 nucleotides. The variable targeting domain can be composed of a DNA sequence, a RNA sequence, a modified DNA sequence, a modified RNA sequence, or any combination thereof.

The term “Cas endonuclease recognition domain” or “CER domain” (of a guide polynucleotide) is used interchangeably herein and includes a nucleotide sequence that interacts with a Cas endonuclease polypeptide. A CER domain comprises a (trans-acting) tracrNucleotide mate sequence followed by a tracrNucleotide sequence. The CER domain can be composed of a DNA sequence, a RNA sequence, a modified DNA sequence, a modified RNA sequence (see for example E1S20150059010A1, published 26 Feb. 2015), or any combination thereof.

As used herein, the terms “guide polynucleotide/Cas endonuclease complex”, “guide polynucleotide/Cas endonuclease system”, “guide polynucleotide/Cas complex”, “guide polynucleotide/Cas system” and “guided Cas system,” “Polynucleotide-guided endonuclease”, “PGEN” are used interchangeably herein and refer to at least one guide polynucleotide and at least one Cas endonuclease, that are capable of forming a complex, wherein said guide polynucleotide/Cas endonuclease complex can direct the Cas endonuclease to a DNA target site, enabling the Cas endonuclease to recognize, bind to, and optionally nick or cleave (introduce a single or double-strand break) the DNA target site. A guide polynucleotide/Cas endonuclease complex herein can comprise Cas protein(s) and suitable polynucleotide component(s) of any of the known CRISPR systems (Horvath and Barrangou, 2010, Science 327: 167-170; Makarova et al. 2015, Nature Reviews Microbiology Vol. 13: 1-15; Zetsche et al. , 2015, Cell 163, 1-13; Shmakov et al., 2015, Molecular Cell 60, 1-13).

The terms “guide RNA/Cas endonuclease complex”, “guide RNA/Cas endonuclease system”, “guide RNA/Cas complex”, “guide RNA/Cas system”, “gRNA/Cas complex”, “gRNA/Cas system”, “RNA-guided endonuclease” , “RGEN” are used interchangeably herein and refer to at least one RNA component and at least one Cas endonuclease that are capable of forming a complex, wherein said guide RNA/Cas endonuclease complex can direct the Cas endonuclease to a DNA target site, enabling the Cas endonuclease to recognize, bind to, and optionally nick or cleave (introduce a single or double-strand break) the DNA target site.

The term “transposon”, as used herein, refers to a polynucleotide (or nucleic acid segment), which may be recognized by a transposase or an integrase enzyme and which is a component of a functional nucleic acid-protein complex (e.g., a transpososome) capable of transposition. The term “transposase” as used herein refers to an enzyme, which is a component of a functional nucleic acid-protein complex capable of transposition and which mediates transposition. The transposase may comprise a single protein or comprise multiple protein sub-units. A transposase may be an enzyme capable of forming a functional complex with a transposon end or transposon end sequences. The term “transposase” may also refer in certain embodiments to integrases. The expression “transposition reaction” used herein refers to a reaction wherein a transposase inserts a donor polynucleotide sequence in or adjacent to an insertion site on a target polynucleotide. The insertion site may contain a sequence or secondary structure recognized by the transposase and/or an insertion motif sequence where the transposase cuts or creates staggered breaks in the target polynucleotide into which the donor polynucleotide sequence may be inserted. Exemplary components in a transposition reaction include a transposon, comprising the donor polynucleotide sequence to be inserted, and a transposase or an integrase enzyme. The term, “transposon end sequence” as used herein refers to the nucleotide sequences at the distal ends of a transposon. The transposon end sequences may be responsible for identifying the donor polynucleotide for transposition. The transposon end sequences may be the DNA sequences the transpose enzyme uses in order to form transpososome complex and to perform a transposition reaction.

The terms “target site”, “target sequence”, “target site sequence,” target DNA”, “target locus”, “genomic target site”, “genomic target sequence”, “genomic target locus” and “protospacer”, are used interchangeably herein and refer to a polynucleotide sequence such as, but not limited to, a nucleotide sequence on a chromosome, episome, a locus, or any other DNA molecule in the genome (including chromosomal, chloroplastic, mitochondrial DNA, plasmid DNA) of a cell, at which a guide polynucleotide/Cas endonuclease complex can recognize, bind to, and optionally nick or cleave. The target site can be an endogenous site in the genome of a cell, or alternatively, the target site can be heterologous to the cell and thereby not be naturally occurring in the genome of the cell, or the target site can be found in a heterologous genomic location compared to where it occurs in nature.

As used herein, terms “endogenous target sequence” and “native target sequence” are used interchangeable herein to refer to a target sequence that is endogenous or native to the genome of a cell and is at the endogenous or native position of that target sequence in the genome of the cell. An “artificial target site” or “artificial target sequence” are used interchangeably herein and refer to a target sequence that has been introduced into the genome of a cell. Such an artificial target sequence can be identical in sequence to an endogenous or native target sequence in the genome of a cell but be located in a different position (i.e., a non-endogenous or non-native position) in the genome of a cell.

A “protospacer adjacent motif” (PAM) herein refers to a short nucleotide sequence adjacent to a target sequence (protospacer) that is recognized (targeted) by a guide polynucleotide/Cas endonuclease system described herein. The Cas endonuclease may not successfully recognize a target DNA sequence if the target DNA sequence is not followed by a PAM sequence. The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

An “altered target site”, “altered target sequence”, “modified target site”, “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, (iv) a chemical alteration of at least one nucleotide, or (v) any combination of (i)-(iv).

A “modified nucleotide” or “edited nucleotide” refers to a nucleotide sequence of interest that comprises at least one alteration when compared to its non-modified nucleotide sequence. Such “alterations” include, for example: (i) replacement of at least one nucleotide, (ii) a deletion of at least one nucleotide, (iii) an insertion of at least one nucleotide, (iv) a chemical alteration of at least one nucleotide, or (v) any combination of (i)-(iv).

Methods for “modifying a target site” and “altering a target site” are used interchangeably herein and refer to methods for producing an altered target site.

As used herein, “donor DNA” is a DNA construct that comprises a polynucleotide of interest to be inserted into the target site of a Cas endonuclease.

The term “polynucleotide modification template” includes a polynucleotide that comprises at least one nucleotide modification when compared to the nucleotide sequence to be edited. A nucleotide modification can be at least one nucleotide substitution, addition, or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

A “complex trait locus” includes a genomic locus that has multiple transgenes genetically linked to each other.

The terms “decreased,” “fewer,” “slower” and “increased” “faster” “enhanced” “greater” as used herein refers to a decrease or increase in a property such as efficiency, processivity, or specificity. For example, a decrease in a characteristic may be at least 1%, at least 2%, at least 3%, at least 4%, at least 5%, between 5% and 10%, at least 10%, between 10% and 20%, at least 15%, at least 20%, between 20% and 30%, at least 25%, at least 30%, between 30% and 40%, at least 35%, at least 40%, between 40% and 50%, at least 45%, at least 50%, between 50% and 60%, at least about 60%, between 60% and 70%, between 70% and 80%, at least 75%, at least about 80%, between 80% and 90%, at least about 90%, between 90% and 100%, at least 100%, between 100% and 200%, at least 200%, at least about 300%, at least about 400%) or more lower than the wild type or other control, and an increase may be at least 1%, at least 2%, at least 3%, at least 4%, at least 5%, between 5% and 10%, at least 10%, between 10% and 20%, at least 15%, at least 20%, between 20% and 30%, at least 25%, at least 30%, between 30% and 40%, at least 35%, at least 40%, between 40% and 50%, at least 45%, at least 50%, between 50% and 60%, at least about 60%, between 60% and 70%, between 70% and 80%, at least 75%, at least about 80%, between 80% and 90%, at least about 90%, between 90% and 100%, at least 100%, between 100% and 200%, at least 200%, at least about 300%, at least about 400% or more higher than the wild type or control.

As used herein, the term “before”, in reference to a sequence position, refers to an occurrence of one sequence upstream, or 5′, to another sequence.

Efficiency is a measure of enzyme activity relative to the theoretical limit of diffusion-limited substrate binding to the enzyme (Johnson et al. 2019). Herein the term “efficiency” is used to refer to the steady-state kinetic parameter, k_{cat}/Kₘ, which is the apparent second-order rate constant for substrate binding and conversion to product. Kinetic parameters derived using direct methods as described in Gong et al. 2018, Liu et al. 2020, and Bravo et al, 2022 (herein incorporated by reference in their entirety) are implicitly given. Even though WT Cas9 catalyzes only a single enzyme turnover such that the products of the reaction remain tightly bound to the enzyme, the equations defining k_{cat}/Kₘ are still valid and will be a function of each step in the reaction cycle from substrate binding to the first largely irreversible step. For example, a mutant Cas9 molecule can have about a 50-fold or less, 40-fold or less, 30-fold or less, 20-fold or less, 10-fold or less, 9-fold or less, 8-fold or less, 7-fold or less, 6-fold or less, 5-fold or less, 4-fold or less, 3-fold or less, 2-fold or less, or 1-fold or less decrease in efficiency as compared to its non-mutant (native) counterpart or to another Cas9. A mutant Cas9 can also have a 1-fold or more, 2-fold or more, 3-fold or more, 4-fold or more, 5-fold or more, 6-fold or more, 7-fold or more, 8-fold or more, 9-fold or more, 10-fold or more, 20-fold or more, 30-fold or more, 40-fold or more, or 50-fold or more increase in efficiency as compared to its non-mutant (native) counterpart or another Cas9.

By “specificity” is meant a function of the efficiency of reaction for a desired substrate relative to that for an undesired substrate (Johnson et al. 2019; Liu et al. 2020; Liu et al. ,2019; Gong et al. 2018). Mathematically, efficiency is defined as the ratio of k_{cat}/Kₘ values to the two substrates. For Cas9, specificity is defined as (k_{cat}/Kₘ)_{on-target-DNA}/(k_{cat}/Kₘ)_{off-target-DNA}. For example, a mutant Cas9 molecule can have about a 50-fold or less, or less, 30-fold or less, 20-fold or less, 10-fold or less, 9-fold or less, 8-fold or less, 7-fold or less, 6-fold or less, 5-fold or less, 4-fold or less, 3-fold or less, 2-fold or less, or 1-fold or less decrease in specificity as compared to its non-mutant (native) counterpart or to another Cas9. A mutant Cas9 can also have a 1-fold or more, 2-fold or more, 3-fold or more, 4-fold or more, 5-fold or more, 6-fold or more, 7-fold or more, 8-fold or more, 9-fold or more, 10-fold or more, 20-fold or more, 30-fold or more, 40-fold or more, or 50-fold or more increase in specificity as compared to its non-mutant (native) counterpart or another Cas9.

### General Description

### Cas9 Variants

The structure for Group IIb Cas9 molecules was determined when bound in complex with a gRNA and double-stranded DNA target, in an active (DNA cleavage product state) and inactive (nonproductive state) conformation. This allowed for rational design of enzymes with different properties that facilitate better gene editing. Specifically, sites in the Rec3 clamp domain were identified that interact with the DNA target and can be modified to engineer enzymes with enhanced properties, such as processivity, activity, and/or specificity. These modifications can improve Cas9 on-target activity and fidelity.

Disclosed herein are isolated Cas9 variants with properties that make them superior to those currently known in the art. Specifically, these Cas9 molecules have one or more variations in the Rec3 clamp domain which can increase processivity, specificity, or fidelity of the Cas9 molecule. The Rec3 clamp domain is within the Rec3 domain. This domain is described in detail in Palermo et al, herein incorporated by reference in its entirety. (Palermo, G., Chen, J., Ricci, C., Rivalta, I., Jinek, M., Batista, V, McCammon, J. (2018). Key role of the REC lobe during CRISPR—Cas9 activation by ‘sensing’, ‘regulating’, and ‘locking’ the catalytic HNH domain. Quarterly Reviews of Biophysics, 51, E9).

Also disclosed herein is a method of increasing fidelity, specificity, and/or speed of processivity in a functional Cas9 molecule, the method comprising mutating one or more amino acid residues within a clamp region of Cas9 Recognition Lobe (REC3 clamp), wherein the REC3 clamp is 80% or more identical to SEQ ID NO: 1 , and further wherein the one or more mutations increase fidelity, specificity, and/or speed of processivity of the Cas9 molecule.

By “variant” or “fragment” is meant a functional fragment or functional variant of a native Cas protein, or a protein that shares at least 30%, between 30% and 35%, at least 35%, between 35% and 40%, at least 40%, between 40% and 45%, at least 45%, between 45% and 50%, at least 50%, 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, or at least 99% sequence identity to a parent Cas9 polypeptide. It is noted that “parent” and “native” are referred to alternatively herein, and have the same meaning, which is the naturally occurring Cas9 on which the variant or fragment thereof is based.

Examples of naturally occurring CRISPR-Cas9s include those found in Gasiunas, G., Young, J. K., Karvelis, T. et al. A catalogue of biochemically diverse CRISPR-Cas9 orthologs. Nat Commun 11, 5512 (2020), hereby incorporated by reference in its entirety for its teaching concerning Cas9 variants. It is noted that every CRISPR-Cas9 ortholog mentioned in Gasiunas is contemplated herein.

Specifically contemplated herein are Cas9 variants such as FnCas9 (SEQ ID NO: 1). The Rec3 Clamp of FnCas9 is depicted in SEQ ID NO: 2.

An example of a Rec3 clamp domain from FnCas9 is depicted in SEQ ID NO: 2, but this sequence can vary in other Cas9 molecules. One of skill in the art will appreciate that the Rec3 clamp domain can vary between different wild type Cas9 molecules or different organisms or engineered Cas9 molecules. Specifically, disclosed herein are other functional Rec3 clamp domains (meaning that they are part of the Rec3 domain and also function as clamps). These variants can have 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% homology with SEQ ID NO: 1, or any amount above, below, or between these percentages. Put another way, contemplated herein are variants of SEQ ID NO: 1 with 1, 2, 3, 4, 5, 6, 7, 8, 9, 10, 11, 12, 13, 14, 15, 16, 17, 18, 19, 21, 22, 23, 24, 25, 26, 27, 28, 29, 30, 31, 32, 33, 34, 35, 36, 37, 38, 39, or 40 or more amino acid residues that vary from SEQ ID NO: 1. One of skill in the art is capable of identifying the Rec3 clamp domain in any Cas9 molecule using the methods disclosed herein.

The isolated Cas9 molecule contemplated herein can have at least one mutation in the Rec3 clamp compared to native or engineered Cas9s from which it is derived. For example, the isolated Cas9 variant or fragment can have 1, 2, 3, 4, 5, 6, 7, 8, 9, 10, 11, 12, 13, 14, 15, 16, 17, 18, 19, or 20 or more amino acid variations in the Rec3 clamp domain when compared to the original Cas9. The disclosed Cas9s can also have mutations which are not in the Rec3 clamp domain. For example, the isolated Cas9 variant or fragment thereof can have 1, 2, 3, 4, 5, 6, 7, 8, 9, 10, 11, 12, 13, 14 ,15, 16, 17, 18, 19, or 20 or more amino acid variations in another area of the protein, in addition to the mutation(s) in the Rec3 clamp domain.

By way of specific example, the isolated Cas9 variant or fragment thereof, as disclosed herein, can have at least one, two, three, four, five, six, seven, eight, nine, or ten or more mutations in at least one of the amino acid residues of SEQ ID NO: 2. Even more specifically, such mutations can be in residues N717, Y794, N725, K789, V675, L723, R721, S792, N626, and/or N801 of SEQ ID NO: 2. Mutations in one or more of these residues can increase (or decrease, if that's desirable) fidelity, speed of processivity, or sensitivity of the Cas9 molecule. For example, a mutation can exist in 1, 2, 3, 4, 5, 6, 7, 8, 9, or 10 of these sites. Mutations can exist in other sites as well as these sites.

The isolated Cas9 variant disclosed herein has endonuclease activity. The isolated Cas9 variant can have less, the same, or more endonuclease activity than the native Cas9 from which it is derived. For example, the isolated Cas9 variant can have a 50-fold or less, 40-fold or less, 30-fold or less, 20-fold or less, 10-fold or less, 9-fold or less, 8-fold or less, 7-fold or less, 6-fold or less, 5-fold or less, 4-fold or less, 3-fold or less, 2-fold or less, or 1-fold or less decrease in endonuclease activity as compared to a native Cas9. The isolated Cas9 variant or fragment thereof can also have a 1-fold or more, 2-fold or more, 3-fold or more, 4-fold or more, 5-fold or more, 6-fold or more, 7-fold or more, 8-fold or more, 9-fold or more, 10-fold or more, 20-fold or more, 30-fold or more, 40-fold or more, or 50-fold or more endonuclease activity as compared to a native Cas9.

As discussed above, the isolated Cas9 variant thereof can have at least one improved property when compared to said parent, or native, Cas9 polypeptide. For example, the isolated Cas9 variant or fragment thereof can have improved specificity when compared to said parent Cas9 polypeptide. The term “specificity a function of the efficiency of reaction for a desired substrate relative to that for an undesired substrate. For example, the specificity of the Cas9 variant or fragment thereof can be a 50-fold or less, 40-fold or less, or less, 20-fold or less, 10-fold or less, 9-fold or less, 8-fold or less, 7-fold or less, 6-fold or less, 5-fold or less, 4-fold or less, 3-fold or less, 2-fold or less, or 1-fold or less decrease in specificity as compared to its non-mutant counterpart or to another Cas9. A mutant Cas9 can also have a 1-fold or more, 2-fold or more, 3-fold or more, 4-fold or more, or more, 6-fold or more, 7-fold or more, 8-fold or more, 9-fold or more, 10-fold or more, 20-fold or more, 30-fold or more, 40-fold or more, or 50-fold or more increase in specificity as compared to its non-mutant counterpart or another Cas9. In a specific embodiment, the isolated Cas9 variant or fragment thereof does not have greater than a 10-fold decrease in specificity of as compared to said parent Cas9 polypeptide.

The Cas9 variant can have increased efficiency, also referred to herein as “speed of processivity.” The cleavage rate is not less than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% or the native, or parent, Cas9 from which it was derived. Furthermore, the Cas9 variant can have an increased cleavage rate as compared to the native Cas9. The increase can be an improvement of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 2-fold or more, 3-fold or more, 4-fold or more, 5-fold or more, 6-fold or more, 7-fold or more, 8-fold or more, 9-fold or more, 10-fold or more, 20-fold or more, 30-fold or more, 40-fold or more, or 50-fold or more as compared to the native, or parent, Cas9.

As mentioned above, the isolated Cas9 variant\thereof can have a number of different mutations or variations which can confer a number of different properties. These mutations or variations can be caused by a mutation or variation in the Rec3 clamp domain, as disclosed herein. One of skill in the art can readily identify different other variations in Cas9 that can also be incorporated into the Cas9 variants thereof disclosed herein. For example, Bak et al. (So Young Bak, Youngri Jung, Jinho Park, Keewon Sung, Hyeon-Ki Jang, Sangsu Bae, Seong Keun Kim, Quantitative assessment of engineered Cas9 variants for target specificity enhancement by single-molecule reaction pathway analysis, Nucleic Acids Research, Volume 49, Issue 19, 8 Nov. 2021, Pages 11312-11322, herein incorporated by reference in its entirety for its teaching concerning Cas9 variants) discloses a number of these variants which are contemplated herein.

### Compositions and Kits Comprising Cas9 Variants

The Cas9 molecules discussed herein can be part of a composition or a kit. The composition can comprise other components which can aid in gene editing or other methods that make use of Cas9 (discussed in detail below). This is referred to herein as a “genome editing system” or “gene editing system.” For example, the composition can be a ribonucleoprotein complex, wherein said ribonucleoprotein comprises the isolated Cas9 variant or fragment thereof and a gRNA complex. The gRNA complex can comprise sgRNA, for example.

The gRNA complex can optionally comprise tracrRNA and crRNA. The ribonucleoprotein complex disclosed herein can be capable of recognizing, binding to, and optionally nicking, unwinding, or cleaving all or part of a target sequence.

Also disclosed is an expression vector encoding the isolated Cas9 variant or fragment thereof. These vectors can be part of a composition or a kit. In one embodiment, the vector can further encode a CRISPR molecule. Furthermore, the vector can encode one or more additional elements necessary to form a ribonucleoprotein complex.

Also disclosed herein are host cells, wherein a transformed host cell includes a polynucleotide that encodes for the Cas9 variant. Host cells generally refer cells that can take up exogenous materials, e.g., nucleic acids (such as DNA and RNA), polypeptides, or ribonuclear proteins. Host cells can be, e.g., single cell organisms, such as, e.g., microorganisms, or eukaryotic cells, e.g., yeast cells, mammalian cells (e.g., in culture) etc.

In some embodiments, host cells are prokaryotic cells, e.g., bacterial cells, e.g., E. coli bacteria. Bacterial cells can be Gram-negative or Gram-positive and can belong to the Bacteria (formerly called Eubacteria) domain or the Archaea (formerly called Archaebacteria) domain. Any of these types of bacteria may be suitable as host cells so long as they can be grown in a laboratory setting and can take up exogenous materials.

The host cells can be bacterial cells that are competent or made competent, e.g., in that they are able or made to be able to take up exogenous material such as genetic material. There are a variety of mechanisms by which exogenous materials such as genetic material can be introduced into host cells.

### Methods of Using Cas9

Further disclosed herein are methods of performing gene editing, the method comprising contacting a target site with a Cas9 variant as described herein. This can include the step of providing a polynucleotide encoding Cas9 to a host cell, along with other components necessary for gene editing. These methods can occur in vivo or in vitro. Those of skill in the art will understand and appreciate that there are many methods which can make use of incorporating the Cas9 variants disclosed herein into cells, both in vivo and in vitro. Incorporation of the Cas9 variants disclosed herein into cells can occur in eukaryotic cells or prokaryotic cells. When in vivo, Cas9 can be used, along with a complete gene editing system, to edit genes in an organism such as a mammal, and more specifically, such as a human These applications are discussed below.

In Vitro Applications

In bacteria, there are three general mechanisms, classified as transformation (uptake and incorporation of extracellular nucleic acids such as DNA), transduction (e.g., transfer of genetic material from one cell to another by a plasmid or by a virus that infects the cells, like bacteriophage), and conjugation (direct transfer of nucleic acids between two cells that are temporarily joined). Host cells into which genetic material have been introduced by transformation are generally referred to as “transformed host cells.”

In some embodiments, a polynucleotide encoding a Cas9 variant as disclosed herein is introduced into host cells by transformation. Protocols for transforming host cells are known in the art. For bacterial cells, for example, there are methods based on electroporation, methods based in lipofection, methods based on heat shock, methods based on agitation with glass beads, methods based on chemical transformation, methods based on bombardment with particles coated with exogenous material (such as DNA or RNA, etc.). One of ordinary skill in the art will be able to choose a method based on the art and/or protocols provided by manufacturers of the host cells.

Bacteriophage are viruses that infect bacteria and inject their genomes (and/or any phagemids packaged within the bacteriophage) into the cytoplasm of the bacteria. Generally, bacteriophage replicate within the bacteria, though replication-defective bacteriophage exist.

In some embodiments, a plurality of bacteriophage comprising a phagemid as described herein is incubated together with transformed host cells under conditions that allow the bacteriophage to infect the transformed host cells. The bacteriophage can be replication-competent, e.g., the bacteriophage replicate within the transformed host cells, and the replicated viral particles are released as virions in the culture medium, allowing re-infection of other host cells by bacteriophage.

Methods of the present disclosure can comprise, after the step of providing a plurality of bacteriophage comprising a phagemid that encodes a first selection agent and includes a DNA target site, a step of incubating transformed host cells (into which the polynucleotide encoding the Cas9 variant was introduced) together with a plurality of bacteriophage under culture conditions such that the plurality of bacteriophage infect the transformed host cells. Generally, these conditions are conditions in which expression of the first selection agent confers either a survival disadvantage or a survival advantage, depending on the embodiment.

In certain embodiments, the culture conditions are competitive culture conditions. “Competitive culture conditions” refers to conditions in which a population of organisms (e.g., host cells) is grown together and must compete for the same limited resources, for example, nutrients, oxygen, etc.

Host cells can be incubated in an environment in which there is no or little input of new nutrients. For example, host cells can be incubated in an environment in which there is no or little input of new oxygen, e.g., in sealed containers such as flasks.

Additionally or alternatively, host cells can be incubated in an culture medium that is well-mixed throughout the period of incubation, e.g., a shaking liquid culture. Generally, under such well-mixed conditions, the host cells have similar nutritional requirements and will be in competition for nutrients and/or oxygen (in the case of aerobic organisms) as the nutrients and/or oxygen become depleted by the growing population.

Additionally or alternatively, host cells can be incubated at an approximately constant temperature, e.g., at a temperature most suitable for the type of host cell. For example, for certain bacterial species including E. coli, host cells are typically incubated at a temperature that is around 37° C.

Host cells can be incubated in a liquid culture that is shaken. This shaking is typically vigorous enough to prevent uneven distribution of nutrients and/or settling of some host cells at the bottom of the culture. For example, host cells can be shaken at least 100 rpm (rotations per minute), at least 125 rpm, at least 150 rpm, at least 175 rpm, at least 200 rpm, at least 225 rpm, at least 250 rpm, at least 275 rpm, or at least 300 rpm. In some embodiments, host cells are shaken at between 100 rpm and 400 pm, e.g., between 200 and 350 rpm, e.g., at approximately 300 rpm.

Host cells can be incubated for a period of time before the plurality of bacteriophage is introduced into the culture. This period of time can allow, for example, the host cell population to recover from being in storage and/or to reach a particular ideal density before introduction of the plurality of bacteriophage. During this period of time before the plurality of bacteriophage is introduced, a selection pressure may be used, or it may not be used.

Culture conditions can comprise, e.g., continuous incubation of the host cells together with the bacteriophage over a period of time, e.g., at least 4 hours, at least 8 hours, at least 12 hours, or at least 16 hours. Additionally or alternatively, culture conditions can comprise continuous incubation of the host cells together with the bacteriophage until the growth of the host cells is saturated.

Culture conditions can allow continuous infection of the host cells by bacteriophage. That is, host cells are infect and re-infected continuously (if they survive) during the incubation period.

In Vivo Applications

Disclosed herein are methods of delivering the Cas9 variants disclosed herein to subjects in need thereof. The Cas9 variant can be part of a gene editing system, as described herein. Therefore, disclosed herein is a method of modifying an organism to produce a non-naturally occurring product, or a naturally occurring product in a non-naturally occurring amount, the method comprising contacting a target site of one or more genes within the genome of the organism with an isolated Cas9 variant as described herein.

Also disclosed herein is a method of treating a subject with a disease or disorder which is treatable with gene editing, the method comprising contacting a target site of one or more genes in need of editing within the genome of the subject with an isolated Cas9 variant or a fragment thereof as described herein. This method can be used in a manner which effectively treats said disease or disorder. Contemplated herein are methods wherein the subject is an embryo.

Various diseases and/or conditions can be treated using the methods disclosed herein. For example, a single gene mutation can be treated. Various examples include, but are not limited to, cystic fibrosis, sickle cell disease, Fragile X syndrome, and muscular dystrophy.

In other embodiments, the disease and/or condition includes a dominant mutation. In various embodiments, dominance is characterized by toxic gain of function, loss of function and/or haploinsufficiency. Various examples include amyotrophic lateral sclerosis (ALS), Huntington's disease, neurofibromatosis type 1 and 2, Marfan syndrome, nonpolyposis colorectal cancer, Von Willebrand disease, among many others. In other embodiments, the disease and/or condition including a dominant mutation is retinitis pigmentosa (RP).

In other embodiments, treating the mammal for the disease and/or condition includes in vivo generation of a double stranded break (DSB) in a population of cells in the mammal. In some embodiments, a single stranded break occurs (SSB). In other embodiments, treating the disease and/or conditions includes in vivo homologous recombination (HR) of a DSB. In other embodiments, HR includes non-homologous end joining (NHEJ) introducing missense or nonsense of a protein expressed at the locus. In other embodiments, HR includes homology directed repair (HDR) introducing co-administered template DNA. In other embodiments, the co-administered template DNA is cognate to a wild-type genetic sequence. In other embodiments, the disease and/or condition includes a recessive mutation. In some embodiments, the HR results in an alteration that is an indel. In some embodiments, the HR results in an alteration causing reduced expression of the target polynucleotide sequence. In some embodiments, the HR results in an alteration that abrogates expression of a protein and/or polypeptide from the target polynucleotide sequences. In some embodiments, the alteration results in a knock out of the target polynucleotide sequence. In some embodiments, the HR results in an alteration that adjusts the target polynucleotide sequence from an undesired sequence to a desired sequence. In some embodiments, the alteration is a homozygous alteration. In some embodiments, each alteration is a homozygous alteration. In various embodiments, a quantity of stem cells, or cells differentiated from stem cells, are administered simultaneously or sequentially. Such cells can include autologous cells, including cells with alteration of a target polynucleotide sequence in the cell or cells via the described methods and compositions.

Nucleic acids encoding the various elements of a genome editing system according to the present disclosure can be administered to subjects or delivered into cells by known methods or as described herein. For example, DNA encoding an RNA-guided nuclease (e.g., an RNA-guided nuclease variant described herein) and/or encoding a gRNA, as well as donor template nucleic acids can be delivered by, e.g., vectors (e.g., viral or non-viral vectors), non-vector based methods (e.g., using naked DNA or DNA complexes), or a combination thereof.

Nucleic acids encoding genome editing systems or components thereof can be delivered directly to cells as naked DNA or RNA (e.g., mRNA), for instance by means of transfection or electroporation, or may be conjugated to molecules (e.g., N-acetylgalactosamine) promoting uptake by the target cells (e.g., erythrocytes, HSCs). Nucleic acid vectors may also be used.

Nucleic acid vectors can comprise one or more sequences encoding genome editing system components, such as an RNA-guided nuclease (e.g., an RNA-guided nuclease variant described herein), a gRNA and/or a donor template. A vector can also comprise a sequence encoding a signal peptide (e.g., for nuclear localization, nucleolar localization, or mitochondrial localization), associated with (e.g. inserted into, fused to) a sequence coding for a protein. As one example, a nucleic acid vectors can include a Cas9 coding sequence that includes one or more nuclear localization sequences (e.g., from SV40).

The nucleic acid vector can also include any suitable number of regulatory/control elements, e.g., promoters, enhancers, introns, polyadenylation signals, Kozak consensus sequences, or internal ribosome entry sites (IRES). These elements are well known in the art, and are described in Cotta-Ramusino.

Nucleic acid vectors according to this disclosure include recombinant viral vectors. Other viral vectors known in the art may also be used. In addition, viral particles can be used to deliver genome editing system components in nucleic acid and/or peptide form. For example, “empty” viral particles can be assembled to contain any suitable cargo. Viral vectors and viral particles can also be engineered to incorporate targeting ligands to alter target tissue specificity.

In addition to viral vectors, non-viral vectors can be used to deliver nucleic acids encoding genome editing systems according to the present disclosure. One important category of non-viral nucleic acid vectors are nanoparticles, which may be organic or inorganic. Nanoparticles are well known in the art. Any suitable nanoparticle design may be used to deliver genome editing system components or nucleic acids encoding such components. For instance, organic (e.g. lipid and/or polymer) nanoparticles may be suitable for use as delivery vehicles in certain embodiments of this disclosure.

Non-viral vectors optionally include targeting modifications to improve uptake and/or selectively target certain cell types. These targeting modifications can include e.g., cell specific antigens, monoclonal antibodies, single chain antibodies, aptamers, polymers, sugars (e.g., N-acetylgalactosamine (GalNAc)), and cell penetrating peptides. Such vectors also optionally use fusogenic and endosome-destabilizing peptides/polymers, undergo acid-triggered conformational changes (e.g., to accelerate endosomal escape of the cargo), and/or incorporate a stimuli-cleavable polymer, e.g., for release in a cellular compartment. For example, disulfide-based cationic polymers that are cleaved in the reducing cellular environment can be used.

In certain embodiments, one or more nucleic acid molecules (e.g., DNA molecules) other than the components of a genome editing system, e.g., the RNA-guided nuclease component and/or the gRNA component described herein, are delivered. In an embodiment, the nucleic acid molecule is delivered at the same time as one or more of the components of the Genome editing system are delivered. In an embodiment, the nucleic acid molecule is delivered before or after (e.g., less than about 30 minutes, 1 hour, 2 hours, 3 hours, 6 hours, 9 hours, 12 hours, 1 day, 2 days, 3 days, 1 week, 2 weeks, or 4 weeks) one or more of the components of the genome editing system are delivered. In an embodiment, the nucleic acid molecule is delivered by a different means than one or more of the components of the genome editing system, e.g., the RNA-guided nuclease component and/or the gRNA component, are delivered. The nucleic acid molecule can be delivered by any of the delivery methods described herein. For example, the nucleic acid molecule can be delivered by a viral vector, e.g., an integration-deficient lentivirus, and the RNA-guided nuclease molecule component and/or the gRNA component can be delivered by electroporation, e.g., such that the toxicity caused by nucleic acids (e.g., DNAs) can be reduced. In an embodiment, the nucleic acid molecule encodes a therapeutic protein, e.g., a protein described herein. In an embodiment, the nucleic acid molecule encodes an RNA molecule, e.g., an RNA molecule described herein.

Route of Administration

Genome editing systems, or cells altered or manipulated using such systems, which include the Cas9 variants disclosed herein, can be administered to subjects by any suitable mode or route, whether local or systemic. Systemic modes of administration include oral and parenteral routes. Parenteral routes include, by way of example, intravenous, intramarrow, intrarterial, intramuscular, intradermal, subcutaneous, intranasal, and intraperitoneal routes. Components administered systemically may be modified or formulated to target, e.g., HSCs, hematopoietic stem/progenitor cells, or erythroid progenitors or precursor cells.

Local modes of administration include, by way of example, intramarrow injection into the trabecular bone or intrafemoral injection into the marrow space, and infusion into the portal vein. In an embodiment, significantly smaller amounts of the components (compared with systemic approaches) may exert an effect when administered locally (for example, directly into the bone marrow) compared to when administered systemically (for example, intravenously). Local modes of administration can reduce or eliminate the incidence of potentially toxic side effects that may occur when therapeutically effective amounts of a component are administered systemically.

Administration may be provided as a periodic bolus (for example, intravenously) or as continuous infusion from an internal reservoir or from an external reservoir (for example, from an intravenous bag or implantable pump). Components may be administered locally, for example, by continuous release from a sustained release drug delivery device.

In addition, components may be formulated to permit release over a prolonged period of time. A release system can include a matrix of a biodegradable material or a material which releases the incorporated components by diffusion. The components can be homogeneously or heterogeneously distributed within the release system. A variety of release systems may be useful, however, the choice of the appropriate system will depend upon rate of release required by a particular application. Both non-degradable and degradable release systems can be used. Suitable release systems include polymers and polymeric matrices, non-polymeric matrices, or inorganic and organic excipients and diluents such as, but not limited to, calcium carbonate and sugar (for example, trehalose). Release systems may be natural or synthetic. However, synthetic release systems are preferred because generally they are more reliable, more reproducible and produce more defined release profiles. The release system material can be selected so that components having different molecular weights are released by diffusion through or degradation of the material.

Representative synthetic, biodegradable polymers include, for example: polyamides such as poly(amino acids) and poly(peptides); polyesters such as poly(lactic acid), poly(glycolic acid), poly(lactic-co-glycolic acid), and poly(caprolactone); poly(anhydrides); polyorthoesters; polycarbonates; and chemical derivatives thereof (substitutions, additions of chemical groups, for example, alkyl, alkylene, hydroxylations, oxidations, and other modifications routinely made by those skilled in the art), copolymers and mixtures thereof. Representative synthetic, non-degradable polymers include, for example: polyethers such as poly(ethylene oxide), poly(ethylene glycol), and poly(tetramethylene oxide); vinyl polymers-polyacrylates and polymethacrylates such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose, and various cellulose acetates; polysiloxanes; and any chemical derivatives thereof (substitutions, additions of chemical groups, for example, alkyl, alkylene, hydroxylations, oxidations, and other modifications routinely made by those skilled in the art), copolymers and mixtures thereof.

Poly(lactide-co-glycolide) microsphere can also be used. Typically the microspheres are composed of a polymer of lactic acid and glycolic acid, which are structured to form hollow spheres. The spheres can be approximately 15-30 microns in diameter and can be loaded with components described herein.

Skilled artisans will appreciate that different components of genome editing systems can be delivered together or separately and simultaneously or nonsimultaneously. Separate and/or asynchronous delivery of genome editing system components may be particularly desirable to provide temporal or spatial control over the function of genome editing systems and to limit certain effects caused by their activity.

Different or differential modes as used herein refer to modes of delivery that confer different pharmacodynamic or pharmacokinetic properties on the subject component molecule, e.g., a RNA-guided nuclease molecule, gRNA, template nucleic acid, or payload. For example, the modes of delivery can result in different tissue distribution, different half-life, or different temporal distribution, e.g., in a selected compartment, tissue, or organ.

Some modes of delivery, e.g., delivery by a nucleic acid vector that persists in a cell, or in progeny of a cell, e.g., by autonomous replication or insertion into cellular nucleic acid, result in more persistent expression of and presence of a component. Examples include viral, e.g., AAV or lentivirus, delivery.

SEQUENCES
SEQ ID NO: 1: Full length FnCas9:
GSHMNFKILPIAIDLGVKNTGVFSAFYQKGTSLERLDNKNGKVYELSKDSY
TLLMNNRTARRHQRRGIDRKQLVKRLFKLIWTEQLNLEWDKDTQQAISFLF
NRRGFSFITDGYSPEYLNIVPEQVKAILMDIFDDYNGEDDLDSYLKLATEQ
ESKISEIYNKLMQKILEFKLMKLCTDIKDDKVSTKTLKEITSYEFELLADY
LANYSESLKTQKFSYTDKQGNLKELSYYHHDKYNIQEFLKRHATINDRILD
TLLTDDLDIWNFNFEKFDFDKNEEKLQNQEDKDHIQAHLHHFVFAVNKIKS
EMASGGRHRSQYFQEITNVLDENNHQEGYLKNFCENLHNKKYSNLSVKNLV
NLIGNLSNLELKPLRKYFNDKIHAKADHWDEQKFTETYCHWILGEWRVGVK
DQDKKDGAKYSYKDLCNELKQKVTKAGLVDFLLELDPCRTIPPYLDNNNRK
PPKCQSLILNPKFLDNQYPNWQQYLQELKKLQSIQNYLDSFETDLKVLKSS
KDQPYFVEYKSSNQQIASGQRDYKDLDARILQFIFDRVKASDELLLNEIYF
QAKKLKQKASSELEKLESSKKLDEVIANSQLSQILKSQHTNGIFEQGTFLH
LVCKYYKQRQRARDSRLYIMPEYRYDKKLHKYNNTGRFDDDNQLLTYCNHK
PRQKRYQLLNDLAGVLQVSPNFLKDKIGSDDDLFISKWLVEHIRGFKKACE
DSLKIQKDNRGLLNHKINIARNTKGKCEKEIFNLICKIEGSEDKKGNYKHG
LAYELGVLLFGEPNEASKPEFDRKIKKFNSIYSFAQIQQIAFAERKGNANT
CAVCSADNAHRMQQIKITEPVEDNKDKIILSAKAQRLPAIPTRIVDGAVKK
MATILAKNIVDDNWQNIKQVLSAKHQLHIPIITESNAFEFEPALADVKGKS
LKDRRKKALERISPENIFKDKNNRIKEFAKGISAYSGANLTDGDFDGAKEE
LDHIIPRSHKKYGTLNDEANLICVTRGDAKNKGNRIFCLRDLADNYKLKQF
ETTDDLEIEKKIADTIWDANKKDFKFGNYRSFINLTPQEQKAFRHALFLAD
ENPIKQAVIRAINNRNRTFVNGTQRYFAEVLANNIYLRAKKENLNTDKISF
DYFGIPTIGNGRGIAEIRQLYEKVDSDIQAYAKGDKPQASYSHLIDAMLAF
CIAADEHRNDGSIGLEIDKNYSLYPLDKNTGEVFTKDIFSQIKITDNEFSD
KKLVRKKAIEGFNTHRQMTRDGIYAENYLPILIHKELNEVRKGYTWKNSEE
IKIFKGKKYDIQQLNNLVYCLKFVDKPISIDIQISTLEELRNILTTNNIAA
TAEYYYINLKTQKLHEYYIENYNTALGYKKYSKEMEFLRSLAYRSERVKIK
SIDDVKQVLDKDSNFIIGKITLPFKKEWQRLYREWQNTTIKDDYEFLKSFF
NVKSITKLHKKVRKDFSLPISTNEGKFLVKRKTWDNNFIYQILNDSDSRAD
GTKPFIPAFDISKNEIVEAIIDSFTSKNIFWLPKNIELQKVDNKNIFAIDT
SKWFEVETPSDLRDIGIATIQYKIDNNSRPKVRVKLDYVIDDDSKINYFMN
HSLLKSRYPDKVLEILKQSTIIEFESSGENKTIKEMLGMKLAGIYNETSNN
SEQ ID NO: 2: REC3 CLAMP of FnCas9:
NHKPRQKRYQLLNDLAGVLQVSPNFLKDKIGSDDDLFISKWLVEHIRGFKK
ACEDSLKIQKDNRGLLNHKINIARNTKGKCEKEIFNLICKIEGSEDKKGNY
KHGLAYELGVLLFGEPNEASKPEFDRKIKKFNSIYSFAQIQQIAFAERKGN
AN

What is claimed is:

1. A method of increasing fidelity, specificity, and/or speed of processivity in a functional Cas9 molecule, the method comprising mutating one or more amino acid residues within a clamp region of Cas9 Recognition Lobe (REC3 clamp), wherein the REC3 clamp is 80% or more identical to SEQ ID NO: 2, and further wherein the one or more mutations increase fidelity, specificity, and/or speed of processivity of the Cas9 molecule.

2. The method of claim 1, wherein the one or more mutated amino acid residues are selected from the group comprising N717, Y794, N725, K789, V675, L723, R721, S792, N626, and N801 of SEQ ID NO: 2.

3. The method of claim 1, wherein at least two amino acid residues of SEQ ID NO: 2 are mutated.

4. The method of claim 3, wherein the two or more mutated amino acid residues are selected from the group comprising N717, Y794, N725, K789, V675, L723, R721, S792, N626, and N801 of SEQ ID NO: 2.

5. The method of claim 1, wherein at least three amino acid residues of SEQ ID NO: 1 are mutated.

6. The method of claim 5, wherein the three or more mutated amino acid residues are selected from the group N717, Y794, N725, K789, V675, L723, R721, S792, N626, and N801 of SEQ ID NO: 2.

7. The method of claim 1, wherein at least four amino acid residues of SEQ ID NO: 1 are mutated.

8. The method of claim 7, wherein the four or more mutated amino acid residues are selected from the group comprising N717, Y794, N725, K789, V675, L723, R721, S792, N626, and N801 of SEQ ID NO: 2.

9. The method of claim 1, wherein at least five amino acid residues of SEQ ID NO: 1 are mutated.

10. The method of claim 9, wherein the five or more mutated amino acid residues are selected from the group comprising N717, Y794, N725, K789, V675, L723, R721, S792, N626, and N801 of SEQ ID NO: 2.

11. The method of any one of claims 1-10, wherein the Cas9 molecule further comprises other engineered mutations which are not in the REC3 clamp.

12. An isolated functional Cas9 variant comprising a REC3 clamp, wherein the REC3 clamp is 80% or more identical to SEQ ID NO: 2, and further wherein the REC3 clamp comprises one or more amino acid mutations which increase fidelity, specificity, and/or speed of processivity compared to a REC3 clamp without said one or more mutations.

13. The isolated functional Cas9 variant of claim 12, wherein the one or more mutated amino acid residues are selected from the group comprising N717, Y794, N725, K789, V675, L723, R721, S792, N626, and N801 of SEQ ID NO: 2.

14. The isolated functional Cas9 variant of claim 12, wherein at least two amino acid residues of SEQ ID NO: 2 are mutated.

15. The isolated functional Cas9 variant of claim 14, wherein the two or more mutated amino acid residues are selected from the group comprising N717, Y794, N725, K789, V675, L723, R721, S792, N626, and N801 of SEQ ID NO: 2.

16. The isolated functional Cas9 variant of claim 12, wherein at least three amino acid residues of SEQ ID NO: 2 are mutated.

17. The isolated functional Cas9 variant of claim 16, wherein the three or more mutated amino acid residues are selected from the group comprising N717, Y794, N725, K789, V675, L723, R721, S792, N626, and N801 of SEQ ID NO: 2.

18. The isolated functional Cas9 variant of claim 12, wherein at least four amino acid residues of SEQ ID NO: 2 are mutated.

19. The isolated functional Cas9 variant of claim 18, wherein the four or more mutated amino acid residues are selected from the group comprising N717, Y794, N725, K789, V675, L723, R721, S792, N626, and N801 of SEQ ID NO: 2.

20. The isolated functional Cas9 variant of claim 12, wherein at least five amino acid residues of SEQ ID NO: 2 are mutated.

21. The isolated functional Cas9 variant of claim 20, wherein the five or more mutated amino acid residues are selected from the group comprising N717, Y794, N725, K789, V675, L723, R721, S792, N626, and N801 of SEQ ID NO: 2.

22. The isolated functional Cas9 variant of any one of claims 12-21, wherein the Cas9 molecule further comprises other engineered mutations which are not in the REC3 clamp.

23. A composition comprising the functional isolated Cas9 variant of any one of claims 12-22.

24. The composition of claim 23, wherein said composition is a ribonucleoprotein complex, wherein said ribonucleoprotein comprises the isolated Cas9 variant or fragment thereof and a gRNA complex.

25. The composition of claim 24, wherein the gRNA complex comprises sgRNA.

26. The composition of claim 24 or 25, wherein the gRNA complex comprises tracrRNA and crRNA.

27. The composition of claim 24, wherein said ribonucleoprotein complex is capable of recognizing, binding to, and optionally nicking, unwinding, or cleaving all or part of a target sequence.

28. An expression vector encoding the isolated functional Cas9 variant of any one of claims 12-22.

29. The expression vector of claim 28, wherein the vector further encodes a CRISPR molecule.

30. The expression vector of claim 29, wherein the vector further encodes one or more additional elements necessary to form a ribonucleoprotein complex.

31. A cell encoding the expression vector of any one of claims 28-30.

32. A method of performing gene editing, the method comprising contacting a target site with a functional Cas9 molecule, wherein the Cas9 molecule comprises a REC3 clamp, wherein the REC3 clamp is 80% or more identical to SEQ ID NO: 2, and further wherein the REC3 clamp comprises one or more amino acid mutations which increase fidelity, specificity, and/or speed of processivity compared to a REC3 clamp without said one or more mutations.

33. The method of claim 32, wherein said target site is in a cell.

34. The method of claim 33, wherein said cell is in an organism.

35. The method of claim 34, wherein the organism is a prokaryote.

36. The method of claim 34, wherein the organism is a eukaryote.

37. The method of claim 36, wherein the eukaryote is a mammal.

38. The method of claim 37, wherein the mammal is a human

39. The method of any one of claims 32-38, wherein said contacting occurs via transfection by a vector encoding said the Cas9 molecule.

40. The method of claim 39, wherein said vector further encodes a CRISPR molecule.

41. The method of claim 39 or 40, wherein the vector encodes elements needed to form a ribonucleoprotein complex from said Cas9 molecule and a CRISPR molecule.

42. The method of claim any one of claims 32-41, wherein Cas9 molecule is part of a ribonucleoprotein complex.

43. The method of claim 42, wherein said ribonucleoprotein complex is introduced into the cell by lipofection or electroporation.

44. The method of any one of claims 32-43, wherein the one or more mutated amino acid residues are selected from the group comprising N717, Y794, N725, K789, V675, L723, R721, S792, N626, and N801 of SEQ ID NO: 2.

45. The method of claim 32, wherein at least two amino acid residues of SEQ ID NO: 2 are mutated.

46. The method of claim 45, wherein the two or more mutated amino acid residues are selected from the group comprising N717, Y794, N725, K789, V675, L723, R721, S792, N626, and N801 of SEQ ID NO: 2.

47. The method of claim 32, wherein at least three amino acid residues of SEQ ID NO: 2 are mutated.

48. The method of claim 47, wherein the three or more mutated amino acid residues are selected from the group comprising N717, Y794, N725, K789, V675, L723, R721, S792, N626, and N801 of SEQ ID NO: 2.

49. The method of claim 32, wherein at least four amino acid residues of SEQ ID NO: 2 are mutated.

50. The method of claim 49, wherein the four or more mutated amino acid residues are selected from the group comprising N717, Y794, N725, K789, V675, L723, R721, S792, N626, and N801 of SEQ ID NO: 2.

51. The method of claim 32, wherein at least five amino acid residues of SEQ ID NO: 2 are mutated.

52. The method of claim 51, wherein the five or more mutated amino acid residues are selected from the group comprising N717, Y794, N725, K789, V675, L723, R721, S792, N626, and N801 of SEQ ID NO: 2.

53. The method of any one of claims 32-52, wherein the Cas9 molecule further comprises other engineered mutations which are not in the REC3 clamp.

54. A method of treating a subject with a disease or disorder which is treatable with gene editing, the method comprising contacting a target site of one or more genes in need of editing within the genome of the subject with a functional Cas9 molecule comprising a REC3 clamp, wherein the REC3 clamp is 80% or more identical to SEQ ID NO: 2, and further wherein the REC3 clamp comprises one or more amino acid mutations which increase fidelity, specificity, and/or speed of processivity compared to a REC3 clamp without said one or more mutations; wherein said Cas9 molecule edits one or more genes in a manner which effectively treats said disease or disorder.

55. The method of claim 54, wherein the subject is an embryo.

56. The method of 54 or 55, wherein said contacting occurs via transfection by a vector encoding said Cas9 molecule.

57. The method of claim 56, wherein said vector further encodes a CRISPR molecule.

58. The method of claim 56 or 57, wherein the vector encodes elements needed to form a ribonucleoprotein complex from said Cas9 and a CRISPR molecule.

59. The method of claim any one of claims 54-58, wherein Cas9 molecule is part of a ribonucleoprotein complex.

60. The method of claim 59, wherein said ribonucleoprotein complex is introduced into the cell by lipofection or electroporation.

61. The method of any one of claims 54-60, wherein the one or more mutated amino acid residues are selected from the group comprising N717, Y794, N725, K789, V675, L723, R721, S792, N626, and N801 of SEQ ID NO: 2.

62. The method of claim 54, wherein at least two amino acid residues of SEQ ID NO: 2 are mutated.

63. The method of claim 62, wherein the two or more mutated amino acid residues are selected from the group comprising N717, Y794, N725, K789, V675, L723, R721, S792, N626, and N801 of SEQ ID NO: 2.

64. The method of claim 54, wherein at least three amino acid residues of SEQ ID NO: 2 are mutated.

65. The method of claim 64, wherein the three or more mutated amino acid residues are selected from the group comprising N717, Y794, N725, K789, V675, L723, R721, S792, N626, and N801 of SEQ ID NO: 2.

66. The method of claim 54, wherein at least four amino acid residues of SEQ ID NO: 2 are mutated.

67. The method of claim 66, wherein the four or more mutated amino acid residues are selected from the group comprising N717, Y794, N725, K789, V675, L723, R721, S792, N626, and N801 of SEQ ID NO: 2.

68. The method of claim 54, wherein at least five amino acid residues of SEQ ID NO: 2 are mutated.

69. The method of claim 68, wherein the five or more mutated amino acid residues are selected from the group comprising N717, Y794, N725, K789, V675, L723, R721, S792, N626, and N801 of SEQ ID NO: 2.

70. The method of any one of claims 54-69, wherein the Cas9 molecule further comprises other engineered mutations which are not in the REC3 clamp.

71. The method of any one of claims 54-70, wherein the subject has a genetic disorder.

72. The method of any one of claims 54-71, wherein the subject has cancer.

73. A method of modifying an organism to produce a non-naturally occurring product, or a naturally occurring product in a non-naturally occurring amount, the method comprising contacting a target site of one or more genes within the genome of the organism with a functional Cas9 molecule comprising a REC3 clamp, wherein the REC3 clamp is 80% or more identical to SEQ ID NO: 2, and further wherein the REC3 clamp comprises one or more amino acid mutations which increase fidelity, specificity, and/or speed of processivity compared to a REC3 clamp without said one or more mutations; wherein said Cas9 molecule edits one or more genes of interest so that the organism produces a non-naturally occurring product, or a naturally occurring product in a non-naturally occurring amount.

74. The method of claim 73, wherein the organism is prokaryotic.

75. The method of claim 74, wherein the organism is eukaryotic.

76. A kit comprising an isolated functional Cas9 molecule comprising a REC3 clamp, wherein the REC3 clamp is 80% or more identical to SEQ ID NO: 2, and further wherein the REC3 clamp comprises one or more amino acid mutations which increase fidelity, specificity, and/or speed of processivity compared to a REC3 clamp without said one or more mutations.

77. The kit of claim 76, wherein the isolated Cas9 molecule thereof is encoded in a vector.

78. The kit of claim 77, wherein the vector further encodes a CRISPR molecule.

79. The kit of any one of claims 76-78, wherein the kit further comprises one or more additional elements necessary to form a ribonucleoprotein complex.


SEQUENCE LISTING (2 sequences)

SEQ ID NO: 1 - protein, 1632 aa, synthetic construct
  REGION 1..1632: note = Full length FnCas9
GSHMNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDAKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 2 - protein, 155 aa, synthetic construct
  REGION 1..155: note = REC3 CLAMP of FnCas9
NHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNAN